(19) United States
(12) Patent Application Publication
(10) Pub. No.: US 2005/0148531 A1
(43) Pub. Date: Jul. 7, 2005

(54) Modulation of gene expression using DNA-DNA hybrids

(76) Inventors: Todd Hauser, Seattle, WA (US); Aaron Loomis, Prairie Du Sac, WI (US); David Hensel, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/847,204

(22) Filed: May 17, 2004

Related U.S. Application Data
(60) Provisional application No. 60/471,056, filed on May 15, 2003.

(51) Int. Cl. A61K 48/00; C12Q 1/68; C07H 21/02; A61K 31/675; C07F 9/6533

(52) U.S. Cl. 514/44; 514/81; 435/6; 435/455; 536/23.1; 544/81

(57) ABSTRACT

The present invention is directed to novel DNA-DNA hybrids comprising a DNA sense strand and a DNA antisense strand. The compounds of the invention, and compositions and arrays comprising the same, may be used for a variety of purposes, including inhibiting gene expression, treating disease and infection, determining the function of genes, and identifying and validating novel drugs and their targets.

### BACKGROUND OF THE INVENTION

1. Field of the Invention

The present invention relates generally to DNA-DNA hybrids and methods of using the same to modulate gene expression.

2. Description of the Related Art

The phenomenon of gene silencing, or inhibiting the expression of a gene, holds significant promise for therapeutic and diagnostic purposes, as well as for the study of gene function itself. Examples of this phenomenon include antisense technology and posttranscriptional gene silencing (PTGS).

However, many problems remain with development of effective antisense and PTGS technologies. For example, DNA antisense oligonucleotides exhibit only short term effectiveness and are usually toxic at the doses required; similarly, the use of antisense RNAs has also proved ineffective due to stability problems. PTGS techniques, meanwhile, have not been demonstrated to work well in higher vertebrates and, therefore, the widespread use of PTGS for functional analysis, therapeutic, and diagnostic purposes is still questionable.

A more recent approach to quelling specific gene activities is RNA interference (RNAi), a term initially coined by Fire and co-workers to describe the observation that double-stranded RNA (dsRNA) can block gene expression when it is introduced into worms (Fire et al., Nature 291:806-811 (1998)).

Since that time, dsRNA has been found capable of suppressing gene activities in a variety of in-vivo systems, including plants (Grant, S. R., Cell 96:303-306 (1999)), Drosophila melanogaster (Kennerdell, J. and Carthew, R., Cell 95:1017-1026 (1998), Misquitta, L. and Paterson, B., Proc. Natl. Acad. Sci. USA 96:1451-1456 (1999), and Pal-Bhadra, M., Bhadra, U., and Birchler, J. A., Cell 99:35-46 (1999)), and Caenorhabditis elegans (Tabara, H., Sarkissian, M., Kelly, W. G., Fleenor, J., Grishok, A., and Timmons, L., Cell 99:123-132 (1999), Ketting, R., Haverkamp, T., van Luenen, H., and Plasterk, R., Cell 99:133-141 (1999), Fire, A., Xu, S., Montgomery, M., Kostas, S., Driver, S., and Mello, C., Nature 391:806-811 (1998) and Grishok, A., Tabara, H., and Mello, C., Science 287:2494-2497 (2000)).

RNAi appears to evoke an intracellular mRNA degradation process, affecting all highly homologous transcripts, called cosuppression (Jorgensen R., Cluster P., English J., Que Q., and Napoli C., Plant Mol Biol 31:957-73 (1996)). Although experiments investigating gene silencing in lower organisms have offered promising results, it is though that they might not be as consistently and successfully applicable to higher organisms such as mammals. In such higher organisms, it is thought that cellular defense mechanisms operate which are triggered by dsRNA, wherein dsRNA activates the interferon response which leads to global shut-off in protein synthesis as well as non-specific mRNA degradation (Marcus, Interferon 5:115-180 (1983)). This can lead to cell death (Lee & Esteban, Virology 199:491-496 (1994)) and hence prevent selective gene inhibition.

Experiments which have demonstrated the ability of dsRNA to inhibit the expression of a target gene in higher organisms have either been in non-mammalian systems, such as zebrafish (Wargelius, A., Ellingsen, S., and Fjose, A., Biochem. Biophys. Res. Commun. 263:156-161 (1999)) and chicks (Hernandez-Hernandez V., Fernandez J., Cardona A., Romero R., Bueno D., Int. J. of Dev. Biology 45:S99-S100 (2001)), or alternatively in mammalian systems such as early embryos where the viral defense mechanisms are not thought to operate.

It has been proposed that the cosuppression effect of RNAi results from the presence of small RNA known also as small interfering RNA (siRNA). More specifically, siRNA have been observed to consist of partially or completely double-stranded RNA molecules approximately 21 to 25 nucleotide bases in length (Zamore P., Tuschl T., Sharp P., and Bartel D., Cell 101:25-33 (2000)). It has been proposed that these siRNA may be generated by an RNA-directed RNA polymerase (RdRp) (Grant supra) and/or a ribonuclease (RNase) (Ketting et al. supra, Bosher, J. M. and Labouesse, M., Nature Cell Biology 2:31-36 (2000) and Zamore, P. D., Tuschl, T., Sharp, P. A., and Bartel, D. P., Cell 101:25-33 (2000)) activity on an aberrant RNA template derived from the transfecting nucleic acids or viral infection, or they may be synthesized or generated by some other means and introduced to the cell, either in vitro or in vivo.

Preliminary experiments transfecting and/or microinjecting synthetic siRNA rather than longer dsRNA molecules which can be processed to give rise to an siRNA, have led to speculation that it might be possible to overcome the problems of the viral defense mechanisms in higher organisms (Elbashir S., Harborth J., Lendeckel W., Yalcin A., Weber K., and Tuschl T., Nature 411:494-498 (2001)), due to the potential existence of a threshold for the length of dsRNA necessary to activate the cell's defense mechanisms. The size of the synthetic siRNA, and in particular the double-stranded regions in them, may be small enough that they are below this threshold and hence do not activate the defense mechanisms.

The mechanism of RNAi and its inhibitory effect on the target gene has begun to be elucidated (Elbashir S., Lendeckel W., and Tuschl T., Genes & Development 15:188-200 (2002)). Without wishing to be bound to any particular theory, it appears that the initial steps in inhibiting expression involve the generation of a siRNA containing endonuclease complex. The complex then specifically targets the mRNA transcript and involves the exchange of the non-homologous (i.e., non-complementary) strand of the siRNA with the region of sequence homology (complementarity) in the mRNA transcript of the target gene. This in turn is thought to lead to the degradation of the mRNA by the endonuclease complex.

However, while RNAi appears to offer a potential avenue for reducing gene expression, the use of short double-stranded RNA molecules as the catalyst for the directed inhibition of a specific gene has not been demonstrated to work consistently and sufficiently well in higher organisms. Therefore, their widespread use in higher organisms is still questionable. Consequently, there remains a need for an effective and sustained method and composition for the targeted, directed inhibition of gene function in vitro and in vivo in cells of higher vertebrates.

### BRIEF SUMMARY OF THE INVENTION

The present invention provides novel compositions and methods for inhibiting the expression of a target gene in prokaryotes and eukaryotes in vivo and in vitro. In accordance with the present invention, DNA-DNA hybrids are used for reducing the expression of a target gene.

In one embodiment, the invention provides an isolated polynucleotide comprising a double-stranded region consisting of a DNA sense strand and a DNA antisense strand, wherein a blocking agent is attached to the DNA sense strand.

In a related embodiment, the invention provides a DNA-DNA hybrid comprising a DNA sense strand and a DNA antisense strand, wherein a blocking agent is attached to the DNA sense strand or the DNA antisense strand.

In certain embodiments, the DNA antisense strand hybridizes to an mRNA molecule under physiological conditions, while in a related embodiment, the isolated polynucleotide or DNA-DNA hybrid inhibits expression of a polypeptide encoded by the target gene.

In various embodiments, the blocking agent is located on the DNA sense strand and/or the DNA antisense strand. The blocking agent may be located at the 5′ end or the 3′ end of the DNA sense strand or DNA antisense strand, or it may be located at an internal site of the DNA sense or DNA antisense strand. In a related embodiment, the isolated polynucleotide or DNA-DNA hybrid comprises two or more blocking agents, which may be the same as or different from each other.

In a specific embodiment, the blocking agent is a 2,6-Diaminopurine-2′-deoxyriboside, a biotin modifier, an amino modifier, such as aminohexyl, aminododecyl, and trifluoroacetamidehexyl, for example, or 2′OMe. In a related embodiment, the DNA antisense strand is a morpholino.

In one specific embodiment comprising two blocking agents, the first blocking agent is located at the 5′ end of the DNA antisense strand and the second blocking agent is located at the 5′ end of the DNA antisense strand. In various embodiments, the first and second blocking agents are amino modifiers, the first and second blocking agents are biotin modifiers, or one of the blocking agents is an amino modifiers and the other blocking agent is a biotin modifier.

In one embodiment, an isolated polynucleotide or DNA-DNA hybrid of the invention is between 17 and 30 nucleotides in length.

In another aspect, the invention provides an array comprising a plurality of isolated polynucleotides or DNA-DNA hybrids of the invention.

The present invention provides methods for using an isolated polynucleotide or DNA-DNA hybrid of the invention to inhibit or reduce the expression of a target gene. Accordingly, the present invention also relates to DNA-DNA hybrid technology as a powerful new strategy for applications including, without limitation, gene function analysis, the high throughput screening of gene functions (e.g., based on microarray analysis), gene therapy, the suppression of cancer-related genes, the prevention and treatment of microbe-related genes, the study of candidate molecular pathways with systematic knock out of involved molecules, and the validation of targets for and the development of drugs and pharmaceutical agents.

In one embodiment, the invention provides a method for reducing the expression of a gene, comprising introducing an isolated polynucleotide or DNA-DNA hybrid of the invention into a cell. The cell may be plant, animal, protozoan, viral, bacterial, or fungal. In one embodiment, the cell is mammalian.

In various embodiments of methods of the invention, the isolated polynucleotide or DNA-DNA hybrid, or individual molecules thereof, are introduced directly into the cell or introduced extracellularly by a means sufficient to deliver the isolated polynucleotide or DNA-DNA hybrid into the cell.

In a related aspect, the invention provides a method for treating a disease, comprising introducing an isolated polynucleotide or DNA-DNA hybrid of the invention into a cell, wherein overexpression of the mRNA is associated with the disease. In one embodiment, the disease is a cancer.

In a related embodiment, the invention provides a method of treating an infection in a patient, comprising introducing into the patient an isolated polynucleotide of DNA-DNA hybrid of the invention, wherein the isolated polynucleotide entry, replication, integration, transmission, or maintenance of an infective agent.

The invention further provides a method for identifying a function of a gene, comprising introducing into a cell an isolated polynucleotide or DNA-DNA hybrid of the invention, wherein the isolated polynucleotide or the DNA-DNA hybrid inhibits expression of the gene and determining the effect on a characteristic of the cell.

In one embodiment, methods of the invention are utilized during high throughput screening.

### BRIEF DESCRIPTION OF THE SEVERAL VIEWS OF THE DRAWING(S)

FIG. 1 depicts a schematic drawing of an exemplary DNA-DNA hybrid of the invention, in which the DNA sense and antisense strands of the hybrid each incorporate a 2,6-Diaminopurine-2′-deoxyriboside chemically linked to the 5′ end of each DNA molecule (SEQ ID NOS: 1-2).

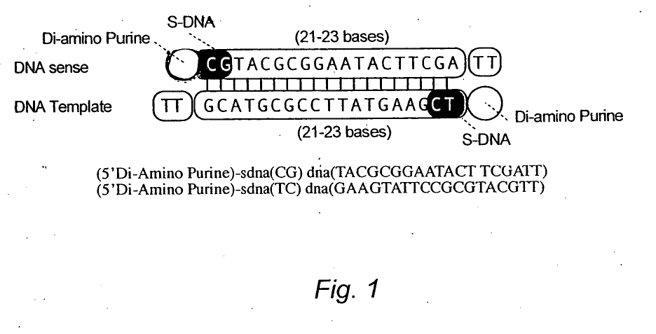

### DETAILED DESCRIPTION OF THE INVENTION

The present invention provides novel compositions and methods for inhibiting the expression of a target gene in prokaryotes and eukaryotes in vivo and in vitro.

Without being bound by any particular theory, the method of this invention is potentially based on the phenomenon of RNA interference (RNAi) as a pathway for inhibiting the expression of a gene. Accordingly, the present invention provides a method of mediating RNAi in a cell or organism. As used herein the phrase “mediating RNAi” refers to (indicates) the ability to distinguish which mRNA are to be degraded by the RNAi machinery or process. The composition of the present invention interacts with the RNAi machinery such that it directs the machinery to degrade particular mRNAs. In specific embodiments, the present invention provides a composition that is effective to inhibit the expression of the targeted gene in vitro or in vivo. The invention is based, in part, upon the surprising discovery that DNA-DNA hybrids can be recognized by RISC and are capable of mediating RNAi.

The invention provides a suite of related DNA-DNA hybrids comprising a blocking agent, which appears to facilitate recognition of the DNA-DNA hybrid by RISC. Each strand of the DNA-DNA hybrid may be designed to possess the ability to modulate or reduce expression of a target gene, depending upon, in part, the sequence of the strand and the presence or absence of one or more blocking groups within the strand. Accordingly, the invention includes related DNA-DNA hybrids, with the functionality of the hybrid, or individual strands thereof, determined by the optional presence or absence of blocking groups in the DNA sense strand of the hybrid. The ability of the DNA-DNA hybrids of the invention to possess dual functionality offers unique advantages for both design and efficacy. For example, a DNA-DNA hybrid may be designed to target more than one gene, and the increased activity associated with dual functionality reduces the required dosage, thus minimizing deleterious toxicity and non-specific effects.

DNA-DNA Hybrids

In accordance with the present invention, DNA-DNA hybrids are used for inhibiting the expression of one or more target genes. Inhibition of target genes is specific in that a nucleotide sequence from a portion of the target gene is the same as all or part of either strand within the DNA-DNA hybrid. Accordingly, the present invention encompasses a variety of DNA-DNA hybrids, each having a DNA sense strand and a DNA antisense strand, the DNA antisense strand of which comprises a nucleotide sequence with complementarity to an mRNA expressed from a target gene. In one embodiment, the DNA sense strand also comprises a nucleotide sequence with complementarity to an mRNA expressed from a target gene. A complementary nucleotide sequence may be completely complementary to a region of an mRNA. Alternatively, the complementary region may be only a portion of the DNA sense or DNA antisense strand, or it may be less than completely complementary, as long as the strand, or a fragment thereof, is capable of binding to an mRNA or capable of directing degradation of a target mRNA. The mRNA may be transcribed from a gene of any species, including, for example, plant, animal (e.g. mammalian), protozoan, viral, bacterial or fungal.

In one embodiment, the DNA-DNA hybrid is an isolated polynucleotide comprising or consisting of a sense DNA strand and an antisense RNA strand. The DNA sense and antisense strands may be complete complements of each other, or they may be less than completely complementary, as long as the strands hybridize to each other under physiological conditions. Typically, the DNA sense and antisense strands are 17 to 26 nucleotides in length, 17 to 30 nucleotides in length or 18 to 23 nucleotides in length, including integer values within these ranges. The DNA sense and antisense strands of a hybrid may be the same or different lengths. The term isolated refers to a material that is at least partially free from components that normally accompany the material in the material's native state. Isolation connotes a degree of separation from an original source or surroundings. Isolated, as used herein, e.g., related to DNA, refers to a polynucleotide that is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

In one embodiment, the DNA-DNA hybrid comprises or consists of a) a first deoxyribonucleic acid molecule approximately 17 to 26 or 17 to 30 nucleotides in length (including any integer value in-between), capable of hybridizing under physiological conditions to at least a portion of an mRNA molecule, and b) a second deoxyribonucleic acid molecule approximately 17 to 26 or 17 to 30 nucleotides in length (including any integer value in-between) capable of hybridizing under physiological conditions to at least a portion of the first molecule.

One of skill in the art would understand that a wide variety of different DNA-DNA hybrids may be used to target a specific gene or transcript. In certain embodiments, DNA-DNA hybrid molecules, or strands thereof, according to the invention are 17 -26 or 17 -30 nucleotides in length, including each integer in between. In one embodiment, a DNA-DNA hybrid, or a strand thereof, is 21 nucleotides in length. In certain embodiments, DNA-DNA hybrids have 0-7 nucleotide 3′ overhangs or 0-4 nucleotide 5′ overhangs. In one embodiment, a DNA-DNA hybrid molecule has a two nucleotide 3′ overhang. In one embodiment, a DNA-DNA hybrid is 21 nucleotides in length with two nucleotide 3′ overhangs (i.e., they contain a 19 nucleotide complementary region between the sense and antisense strands). In certain embodiments, the overhangs are UU, dTdT, or non-naturally occurring nucleic acid 3′ overhangs. In other embodiments, the DNA-DNA hybrid may have a modified backbone composition, such as, for example, 2′-deoxy-or 2′-O-methyl modifications.

In one embodiment, target sites are selected by scanning the target mRNA transcript sequence for the occurrence of AA dinucleotide sequences. Each AA dinucleotide sequence in combination with the 3′ adjacent approximately 19 nucleotides are potential target sites. In one embodiment, target sites are preferentially not located within the 5′ and 3′ untranslated regions (UTRs) or regions near the start codon (within approximately 75 bases), since proteins that bind regulatory regions may interfere with the binding of the siRNP endonuclease complex (Elshabir, S. et al., Nature 411:494-498 (2001); Elshabir, S. et al., EMBO J. 20:6877-6888 (2001)). In addition, potential target sites may be compared to an appropriate genome database, such as BLAST, available on the NCBI server at www.ncbi.nlm, and potential target sequences with significant homology to other coding sequences eliminated.

In one particular embodiment, DNA-DNA hybrids of the invention possess dual functions, e.g., the DNA sense strand functions as an antisense molecule to inhibit expression of a target gene, and the DNA antisense strand functions as siRNA to direct cleavage of a target mRNA. The DNA sense and DNA antisense strands of the hybrid may target different genes or the same gene. For example, the two strands may target different alleles of a gene, including, e.g., single nucleotide polymorphs (SNPs). In another embodiment, the two strands may target the same gene, particularly if the target gene contains one or more inverted repeat regions, such that one repeat region may be bound by either the DNA sense or DNA antisense strand, while a corresponding inverted repeat region may be bound by the other strand.

In one embodiment, the DNA sense strand of the hybrid comprises additional nucleotides that extend 3′ beyond the DNA antisense strand. The sequence of the additional nucleotides may correspond to or be substantially similar to the same gene being targeted by the DNA antisense strand. Alternatively, the sequence of the additional nucleotides may correspond to a different gene than that being targeted by the DNA antisense strand. In one embodiment, the additional sequence of the DNA sense strand is complementary to the same mRNA being targeted by the DNA antisense strand. In this embodiment, the DNA sense strand and the DNA antisense strand may bind to or target the same or different regions of a target polynucleotide. Accordingly, in one embodiment, the DNA strand comprises a region having the same sequence as the DNA antisense strand, in addition to a region having a complementary sequence to at least a region of the DNA antisense strand.

Without wishing to be bound to a particular theory, it is believed that the DNA sense strand, after being separated from the DNA antisense strand by RISC, may enter the nucleus and function to inhibit transcription or expression of a target gene. For example, the DNA sense strand may function as an antisense molecule by binding to an mRNA, or, alternatively, it may function to inhibit transcription by binding double-stranded DNA to form a triplex. Single-stranded DNA fragments may be used as regulatory molecules to inhibit gene expression. Single DNA strands may bind duplex DNA, thereby forming a collinear triplex molecule and preventing transcription (see, e.g., U.S. Pat. No. 5,176,996 to Hogan et al., which describes methods for making synthetic oligonucleotides that bind to target sites on duplex DNA). Since the DNA-DNA hybrid of the invention is capable of silencing gene expression at two levels, it is more potent than traditional antisense or RNA interference agents, and decreased amounts are needed to reduce gene expression in vivo.

Generally, selection of the appropriate sequence to be included within the DNA sense strand is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. These principles may be applied to the selection of the sequence of the DNA sense strand. Preferred target regions include those regions at or near the AUG translation initiation codon and those sequences which are substantially complementary to 5′ regions of mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

In one embodiment, the DNA-DNA hybrids of the invention comprise a blocking agent. A blocking agent as used herein refers to any moiety that is introduced into or attached to one or both of the strands of the hybrid and functions to inhibit or reduce degradation of the DNA-DNA hybrid, or a strand thereof, under physiological conditions, such as the conditions within a cell. The blocking agent typically reduces degradation by making the hybrid, or a strand thereof, more resistant to nuclease degradation tha DNA-DNA hybrids comprising natural DNA sense and antisense strands. Blocking agents may possess additional functions, including raising or lowering the Tm of binding of the two strands of the DNA-DNA hybrid to each other or of binding of the DNA antisense strand to the target mRNA. In addition, the presence of a blocking agent may facilitate cellular uptake and/or reduce undesired side effects.

In one embodiment, the blocking agent functions to facilitate acceptance of the DNA-DNA duplex by the RNA-induced silencing complex (RISC). Without wishing to bound by any particular theory, it is believed that siRNA fragments are recognized and bound by a complex of host cell enzymes called RISC and that this complex unwinds a short double stranded siRNA into a short single strand. RISC then uses these single-strand siRNAs to identify and target RNA strands in the cell capable of binding the siRNA due to a complementary RNA sequence. When RISC finds an RNA that binds to a fragment it is carrying, an enzyme within RISC cleaves this RNA target. It has been suggested that the use of non-modified nucleic acid on the sense strand can diminish recognition of the duplex by RISC (Tuschl, T., CHEMBIO 2:239-245 (2001)).

One or more blocking agents may be introduced into either or both of the DNA sense and antisense strands of the DNA-DNA hybrid. Accordingly, the invention includes DNA-DNA hybrids with one or more blocking agents in the DNA sense strand, DNA-DNA hybrids with one or more blocking agents in the DNA antisense strand, and DNA-DNA hybrids with one or more blocking agents in both the DNA sense and antisense strands.

Blocking agents may be introduced into any region of the DNA strands, including the 5′ end, the 3′ end, or internally. The skilled artisan would readily appreciate that the site of introduction of a blocking agent depends, in large part, on the characteristics and chemical structure of the particular blocking agent being used.

A variety of different blocking agents are contemplated by the invention. Blocking agents that may be introduced into a DNA-DNA hybrid of the invention include, but are not limited to, phosphate groups, amino modifiers, phosphorothioate groups, deoxyinosine residues, deoxyuridine, halogenated nucleosides, 2′O-Methyl groups, 3′-Glycerol groups, 3′-terminators, 5′-propyne pyrimidines, acrydite, cholesterol labels, inverted dT's, dabcyl, digoxigenin labels, methylated nucleosides, spacer reagents, thiol modifications, fluorescent dyes, and biotin modifiers. Modified oligonucleotides and modifying agents that may be used to introduce a blocking agent into a DNA or RNA strand are widely known and commercially available, e.g., from Qiagen, Operon, Integrated DNA Technologies, Glen Research, and Retrogen, Inc.

In one specific embodiment, the blocking agent is 2,6-diaminopurine. This modified base can form three hydrogen bonds when base-paired with dT and can increase the Tm of short oligos by as much as 1-2° C. per insertion and appears to reduce hybrid degradation. 2,6-diaminopurine can be introduced 5′ or internally. In one aspect of this embodiment, the DNA strand of the hybrid incorporates a 2,6-Diaminopurine-2′-deoxyriboside chemically linked to the 5′ end of the molecule. In another aspect of this embodiment, the DNA strand of the hybrid does not incorporate a 2,6-Diaminopurine-2′-deoxyriboside.

In another exemplary embodiment, the blocking agent is an inverted dT. Inverted dT can be incorporated at the 3′-end of an oligo, leading to a 3′-3′ linkage which inhibits both degradation by 3′ exonucleases and extension by DNA polymerases.

In one embodiment, the blocking agent is 2′-O-Methyl. 2′-O-Methyl RNA is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stable with respect to attack by single-stranded ribonucleases and is typically 5 to 10-fold less susceptible to DNases than DNA. It is commonly used in antisense oligos as a means to increase stability and binding affinity to the target message.

In another exemplary embodiment, the blocking agent is a biotin modification. Biotinylated oligonucleotides have been used in a large number of molecular biology applications including quantification of PCR-amplified sequences, chemiluminescent sequencing, in situ hybridization, solid phase restriction site mapping, single base mutational analysis, genomic walking, and cloning of unknown DNA sequences. Once incorporated, the biotin label can be detected by standard streptavidin-based detection methods. Examples of biotin modification include biotin-TEG, which may be introduced 3′, 5′ or internally, and biotin-dT, which may be introduced internally.

In another embodiment, the blocking agent is phosphorothioate. Phosphorothioate analogues of DNA and RNA have sulphur in place of oxygen as one of the non-bridging ligands bound to the phosphorus. Phosphorothioates have been shown to be more resistant to nuclease degradation than the natural DNA and RNA and still to bind to complementary nucleic acid sequences. Phosphorothioate oligodeoxy-nucleotides have demonstrated their usefulness as antisense molecules inhibiting gene expression and as potential chemotherapeutic agents. Phosphorothioate modification is available at any position in an oligonucleotide and can be used multiple times within a sequence.

The invention also contemplates the use of a morpholino oligo as either strand of the DNA-DNA hybrid. Morpholino oligos are so named because they are assembled from four different Morpholino subunits, each of which contains one of the four genetic Bases (Adenine, Cytosine, Guanine, and Thymine) linked to a 6-membered morpholine ring. Typically, eighteen to 25 subunits of these four subunit types are joined in a specific order by non-ionic phosphorodiamidate intersubunit linkages to give a Morpholino oligo. These Morpholino oligos with their 6-membered morpholine backbone moieties joined by non-ionic linkages may provide better antisense properties than do RNA, DNA, and their analogs having 5-membered ribose or deoxyribose backbone moieties joined by ionic linkages.

In one particular embodiment of this aspect of the invention, the DNA sense strand comprises a blocking group, as described supra. The DNA-DNA hybrid may contain one or more blocking groups at any position, such as, e.g., diamino purine at the 5′ end of the DNA sense strand. However, in certain embodiments, the DNA sense strand of the DNA-DNA hybrid does not comprise a blocking group. It is believed that a blocking group is not necessary to prevent degradation of the DNA sense strand, since the DNA sense strand is largely protected while in the duplex. In another related embodiment, the DNA sense strand comprises a blocking group but does not comprise a phosphorothioate. The lack of phosphorothioate modifications eliminates the toxicity associated with phosphorothioated DNA (S-DNA). In one particular embodiment, the DNA-DNA duplex comprises a blocking group at the 5′ end of the DNA antisense strand but does not include a blocking group at the 5′ end of the DNA sense strand.

In another related embodiment, either or both of the DNA sense and antisense strands of the DNA-DNA hybrid does not contain a blocking agent or contains no blocking agents except for one or more phosphorothioates. Since the sense strand may mediate cosuppression, it may be advantageous to not include a blocking agent on the DNA sense strand, so the DNA sense strand undergoes degradation and cannot cause cosuppression.

In another embodiment, the DNA-DNA hybrid comprises a GC clamp, which functions to reduce degradation of the DNA-DNA hybrid. Since GC rich regions of double-stranded nucleotides melt at higher temperatures than regions that are AT rich, the integrity of the duplex may be protected by incorporating a GC-rich region, or GC clamp, into the duplex. In one embodiment, the GC clamp is at the 5′ end of the DNA-DNA duplex. The GC clamp is typically two nucleotides in length on each complementary strand, although it may be longer, e.g., two to ten nucleotides, ten to twenty nucleotides, twenty to forty nucleotides, or any integer value within these ranges. The GC clamp generally comprises only C and G nucleotides. In one embodiment, the GC clamp comprises a 5′ CG on the DNA sense strand and a 3′ GC on the corresponding DNA antisense strand of the duplex. In another embodiment, the GC clamp comprises a 5′ GC on the DNA sense strand and a 3′ CG on the corresponding DNA antisense strand of the duplex.

Accordingly, the present invention also relates to methods of producing DNA-DNA hybrid molecules, by methods such as chemical synthesis or recombinant techniques, that have the ability to mediate RNAi. This includes methods of isolating, prior to hybridization, DNA molecules obtained by any means, including processing or cleavage of dsDNA; production by chemical synthetic methods; and production by recombinant DNA techniques. These include isolated DNA molecules (partially purified DNA, essentially DNA, synthetic DNA, recombinantly produced DNA), as well as altered DNA that differs from naturally occurring DNA by the addition, substitution and/or alteration of one or more deoxyribonucleotides, such as to the end(s) of the 17-26 or 17-30 nt DNA; by one or more modifications to the phosphate-sugar backbone of the DNA; or by the addition, deletion, substitution and/or alteration of one or more nucleotides, wherein alterations can include addition of non-nucleotide material, such as to the end(s) of the approximately 17 to 26, 17 to 30, or 18-23 nt DNA or internally (at one or more nucleotides of the DNA). Nucleotides in the DNA molecules of the present invention can also comprise non-standard nucleotides, including non-naturally occurring nucleotides or deoxyribonucleotides. The DNA molecules of the DNA-DNA hybrid may be synthesized either in vivo or in vitro. Hybridization of the molecules may be initiated either inside or outside of the cell.

The invention further provides arrays of DNA-DNA hybrids of the invention, including microarrays. Microarrays are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided only that such techniques are amenable to and compatible with the deposition and/or screening of polynucleotide sequences.

Microarrays of the invention are particularly desirable for high throughput analysis of multiple DNA-DNA hybrids. A DNA microarray typically is constructed with discrete region or spots that comprise DNA-DNA hybrids of the invention. Each spot may comprise one or more DNA-DNA hybrids of the invention. Arrays of the invention preferably contain DNA-DNA hybrids at positionally addressable locations on the array surface. Arrays of the invention may be prepared by any method available in the art. For example, the light-directed chemical synthesis process developed by Affymetrix (see, U.S. Pat. Nos. 5,445,934 and 5,856,174) may be used to synthesize biomolecules on chip surfaces by combining solid-phase photochemical synthesis with photolithographic fabrication techniques. The chemical deposition approach developed by Incyte Pharmaceutical uses pre-synthesized cDNA probes for directed deposition onto chip surfaces (see, e.g., U.S. Pat. No. 5,874,554).

Methods of Inhibiting Gene Expression

DNA-DNA hybrids of the invention may be used for a variety of purposes, all related to the ability of the hybrids to inhibit or reduce expression of a target gene. Accordingly, the invention provides methods of reducing expression of one or more target genes comprising introducing a DNA-DNA hybrid of the invention into a cell that contains a target gene or a homolog, variant or ortholog thereof. To effectively reduce expression from the gene, it is understood that the DNA antisense strand, or a fragment thereof, must be capable of binding to an mRNA transcribed from the target gene.

A target gene may be a gene derived from the cell, an endogenous gene, a transgene, or a gene of a pathogen which is present in the cell after transfection thereof. Depending on the particular target gene and the amount of the DNA-DNA hybrid delivered into the cell, the method of this invention may cause partial or complete inhibition of the expression of the target gene. The cell with the target gene may be derived from or contained in any organism (e.g., plant, animal, protozoan, virus, bacterium, or fungus).

Inhibition of the expression of the target gene can be verified by means including but not limited to observing or detecting an absence or observable decrease in the level of protein encoded by a target gene, and/or mRNA product from a target gene, and/or by phenotype associated with expression of the gene, using techniques known to a person skilled in the field of the present invention. Examples of cell characteristics that may be examined to determine the effect caused by introduction of a DNA-DNA hybrid of the invention include, cell growth, apoptosis, cell cycle characteristics, cellular differentiation, and morphology.

In one embodiment of the invention, the level of inhibition of target gene expression (i.e., mRNA expression) is at least 90%, at least 95%, at least 98%, at least 99% or is almost 100%, and hence the cell or organism will in effect have the phenotype equivalent to a so-called “knock out” of a gene. However, in some embodiments, it may be preferred to achieve only partial inhibition so that the phenotype is equivalent to a so-called “knockdown” of the gene. This method of knocking down gene expression can be used therapeutically or for research (e.g., to generate models of disease states, to examine the function of a gene, to assess whether an agent acts on a gene, to validate targets for drug discovery).

The DNA-DNA hybrid, or the individual molecules thereof, may be directly introduced to the cell (i.e., intracellularly), or introduced extracellularly into a cavity, interstitial space, into the circulation of an organism, introduced orally, by bathing an organism in a solution containing the DNA-DNA hybrid, or by some other means sufficient to deliver the hybrid or its component molecules into the cell to mediate RNAi.

Methods of inhibiting gene expression using DNA-DNA hybrids of the invention may be combined with other knockdown and knockout methods, e.g., gene targeting, antisense RNA, ribozymes, double-stranded RNA (e.g., shRNA and siRNA) to further reduce expression of a target gene.

Accordingly, the present invention may also be used for the treatment or prevention of disease. For example, a DNA-DNA hybrid may be introduced into a cancerous cell or tumor and thereby inhibit gene expression of a gene required for maintenance of the carcinogenic/tumorigenic phenotype. To prevent a disease or other pathology, a target gene may be selected which is required for initiation or maintenance of the disease/pathology. Treatment may include amelioration of any symptom associated with the disease or clinical indication associated with the pathology.

A gene derived from any pathogen may be targeted for inhibition. For example, the gene could cause immunosuppression of the host directly or be essential for replication of the pathogen, transmission of the pathogen, or maintenance of the infection. The inhibitory DNA-DNA hybrid may be introduced in cells in vitro or ex vivo and then subsequently placed into an animal to affect therapy, or directly treated by in vivo administration. The invention, therefore, provides methods of gene therapy. For example, cells at risk for infection by a pathogen or already infected cells, particularly human immunodeficiency virus (HIV) infections, may be targeted for treatment by introduction of a DNA-DNA hybrid according to the invention. The target gene might be a pathogen or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, can be envisioned. In addition, the present invention could be used for treatment or development of treatments for cancers of any type.

The invention also includes a method of identifying gene function in an organism comprising the use of a DNA-DNA hybrid to inhibit the activity of a target gene of previously unknown function. Instead of the time consuming and laborious isolation of mutants by traditional genetic screening, functional genomics envisions determining the function of uncharacterized genes by employing the invention to reduce the amount and/or alter the timing of target gene activity. The invention could be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

A simple assay would be to inhibit gene expression according to the partial sequence available from an expressed sequence tag (EST). Functional alterations in growth, development, metabolism, disease resistance, or other biological processes would be indicative of the normal role of the EST's gene product.

The ease with which a DNA-DNA hybrid can be introduced into an intact cell/organism containing the target gene allows the present invention to be used in high throughput screening (HTS). For example, solutions containing DNA-DNA hybrids that are capable of inhibiting the different expressed genes can be placed into individual wells positioned on a microtiter plate as an ordered array, and intact cells/organisms in each well can be assayed for any changes or modifications in behavior or development due to inhibition of target gene activity. The function of the target gene can be assayed from the effects it has on the cell/organism when gene activity is inhibited. In one embodiment, DNA-DNA hybrids of the invention are used for chemocogenomic screening, i.e., testing compounds for their ability to reverse a disease modeled by the reduction of gene expression using a DNA-DNA hybrid of the invention.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can be amplified to produce an RNA, a DNA-DNA hybrid can be introduced to the organism, and whether an alteration in the characteristic is correlated with inhibition can be determined.

The present invention may be useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of a DNA-DNA hybrid at specific times of development and locations in the organism without introducing permanent mutations into the target genome.

If alternative splicing produced a family of transcripts that were distinguished by usage of characteristic exons, the present invention can target inhibition through the appropriate exons to specifically inhibit or to distinguish among the functions of family members. For example, a hormone that contained an alternatively spliced transmembrane domain may be expressed in both membrane bound and secreted forms. Instead of isolating a nonsense mutation that terminates translation before the transmembrane domain, the functional consequences of having only secreted hormone can be determined according to the invention by targeting the exon containing the transmembrane domain and thereby inhibiting expression of membrane-bound hormone.

Also the subject of the present invention is a method of validating whether an agent acts on a gene. In this method, a DNA-DNA hybrid that targets the mRNA to be degraded is introduced into a cell or organism in which RNAi occurs. The cell or organism (which contains the introduced hybrid) is maintained under conditions under which degradation of mRNA occurs, and the agent is introduced into the cell or organism. Whether the agent has an effect on the cell or organism is determined; if the agent has no effect on the cell or organism, then the agent acts on the gene.

The present invention also relates to a method of validating whether a gene product is a target for drug discovery or development. A DNA-DNA hybrid that targets the mRNA that corresponds to the gene for degradation is introduced into a cell or organism. The cell or organism is maintained under conditions in which degradation of the mRNA occurs, resulting in decreased expression of the gene. Whether decreased expression of the gene has an effect on the cell or organism is determined, wherein if decreased expression of the gene has an effect, then the gene product is a target for drug discovery or development.

Also encompassed by the present invention is a method of identifying target sites within an mRNA that are particularly suitable for RNAi, as well as a method of assessing the ability of DNA-DNA hybrids to mediate RNAi.

The present invention is based; in part, upon the surprising discovery that DNA-DNA hybrids comprising a blocking agent are extremely effective in reducing target gene expression, particularly as compared to DNA-DNA hybrids lacking blocking agents and double-stranded RNAs. The mechanism through which the DNA-DNA hybrids of the invention provide such effective reduction in gene expression remains unknown, since the increase in effectiveness appears to exceed the results that would be expected if the blocking agent were functioning only to inhibit degradation of the DNA-DNA hybrid or a strand thereof. Furthermore, the DNA-DNA hybrids of the invention offer additional advantages over traditional dsRNA molecules for siRNA, since the use of DNA-DNA hybrids substantially eliminates the off-target suppression associated with dsRNA molecules.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

The practice of the present invention will employ a variety of conventional techniques of cell biology, molecular biology, microbiology, and recombinant DNA, which are within the skill of the art. Such techniques are fully described in the literature. See, for example, MOLECULAR CLONING: A LABORATORY MANUAL, 2ND ED., ed. by Sambrook, Fritsch, and Maniatis (Cold Spring Harbor Laboratory Press, 1989); and DNA CLONING, VOLUMES I AND II (D. N. Glover ed. 1985).

From the foregoing it will be appreciated that, although specific embodiments of the invention have been described herein for purposes of illustration, various modifications may be made without deviating from the spirit and scope of the invention.

### EXAMPLE 1

### Inhibition of Gene Expression by DNA-DNA Hybrids

The ability of DNA-DNA hybrids of the invention to reduce target gene expression was demonstrated in experiments measuring the expression of the GL2 form of the firefly luciferase in 293-Lux and 3T3-Lux cells (fibroblast cell lines that stably expresses the GL2 form of the firefly luciferase) in the presence or absence of a DNA-DNA hybrid comprising the same region of sequence identical to a portion of the GL2 firefly luciferase gene. The effectiveness of this DNA-DNA hybrid was compared to the effectiveness of a traditional RNA-RNA siRNA duplex comprising a region of sequence identical to a portion of the GL2 firefly luciferase gene.

Table 1 provides the results of an experiment measuring the gene expression of the GL2 form of the firefly luciferase following transfection with one of the following: (1) no DNA-DNA hybrid; (2) 1 nM of the DNA-DNA hybrid; (3) 10 nM of the DNA-DNA hybrid; (4) 1 nM of siRNA duplex; and (5)10 nM of siRNA duplex. The numbers provided represent the percentage of gene expression as compared to expression in control cells having received no DNA-DNA hybrid or siRNA duplex.

TABLE 1
Inhibition of gene expression by a DNA—DNA hybrid

| | 293-lux | 3t3-lux |
|---|---|---|
| siRNA duplex | | |
| 1 nM siRNA = | 92% | 25% |
| 10 nM siRNA = | 43% | 12% |
| vs. | | |
| Si2 Version 3 | | |
| 1 nM xsi2 = | 31% | 15% |
| 10 nMxsi2 = | 19% | 21% |

The results of these experiments demonstrate that the present invention is capable of inhibiting the expression of a target gene and that the method of inhibition is titratable and repeatable in different cells. These results further demonstrate that the DNA-DNA hybrids of the invention provide a greater reduction in target gene expression as compared to a traditional siRNA duplex.

1. An isolated polynucleotide comprising a double-stranded region consisting of a DNA sense strand and a DNA antisense strand, wherein a blocking agent is located on the polynucleotide.

2. The isolated polynucleotide of claim 1, wherein the DNA antisense strand hybridizes to an MRNA molecule under physiological conditions.

3. The isolated polynucleotide of claim 2, wherein the isolated polynucleotide inhibits expression of a polypeptide encoded by the mRNA molecule.

4. The isolated polynucleotide of claim 2, wherein the blocking agent is located on the DNA sense strand.

5. The isolated polynucleotide of claim 4, wherein the blocking agent is located at the 5′ end of the DNA sense strand.

6. The isolated polynucleotide of claim 4, wherein the blocking agent is located at the 3′ end of the DNA sense strand.

7. The isolated polynucleotide of claim 4, wherein the blocking agent is located at an internal site of the DNA sense strand.

8. The isolated polynucleotide of claim 2, wherein the blocking agent is located on the DNA antisense strand.

9. The isolated polynucleotide of claim 2, wherein the blocking agent is 2′OMe.

10. The isolated polynucleotide of claim 8, wherein the DNA antisense strand is a morpholino.

11. The isolated polynucleotide of claim 8, wherein the blocking agent is located at the 5′ end of the DNA antisense strand.

12. The isolated polynucleotide of claim 8, wherein the blocking agent is located at the 3′ end of the DNA antisense strand.

13. The isolated polynucleotide of claim 8, wherein the blocking agent is located at an internal site of the DNA antisense strand.

14. The isolated polynucleotide of claim 2, wherein the blocking agent is a 2,6-Diaminopurine-2′-deoxyriboside.

15. The isolated polynucleotide of claim 2, wherein the blocking agent is an amino modifier.

16. The isolated polynucleotide of claim 15, wherein the amino modifier is selected from the group consisting of: aminohexyl, aminododecyl, and trifluoroacetamidehexyl.

17.-19. (canceled)

20. The isolated polynucleotide of claim 8, comprising a first and a second blocking agent, wherein the first blocking agent is located at the 5′ end of the DNA antisense strand and the second blocking agent is located at the 3′ end of the DNA antisense strand.

21. The isolated polynucleotide of claim 20, wherein the first and second blocking agents are amino modifiers.

22. The isolated polynucleotide of claim 20, wherein the first and second blocking agents are biotin modifiers.

23. The isolated polynucleotide of claim 20, wherein one of the blocking agents is an amino modifier and the other blocking agent is a biotin modifier.

24. The isolated polynucleotide of claim 2, wherein the double-stranded region is between 17 and 30 nucleotides in length.

25. The isolated polynucleotide of claim 24, further comprising a single-stranded region of the DNA sense strand.

26. The isolated polynucleotide of claim 25, wherein the DNA sense strand binds to a target gene under physiological conditions.

27. The isolated polynucleotide of claim 26, wherein the DNA sense strand reduces expression of a target gene.

28. The isolated polynucleotide of claim 27, wherein the DNA antisense strand reduces expression of the target gene.

29. An array comprising a plurality of isolated polynucleotides of claim 1.

30. A DNA-DNA hybrid comprising a DNA sense strand and a DNA antisense strand, wherein a blocking agent is attached to the DNA sense strand or the DNA antisense strand.

31. The DNA-DNA hybrid of claim 30, wherein the DNA antisense strand hybridizes to an mRNA molecule under physiological conditions.

32. The DNA-DNA hybrid of claim 31, wherein the DNA-DNA hybrid inhibits expression of a polypeptide encoded by the MRNA molecule.

33. The DNA-DNA hybrid of claim 30, wherein the blocking agent is selected from the group consisting of 2,6-Diaminopurine-2′-deoxyriboside, amino modifiers, biotin modifiers, 2′OMe and morpholino modifiers.

34. (canceled)

35. The DNA-DNA hybrid of claim 30, wherein the DNA-DNA hybrid is between 17 and 30 nucleotides in length.

36. The DNA-DNA hybrid of claim 30 wherein the DNA antisense strand and the DNA sense strand are each between 17 and 30 nucleotides in length, wherein the DNA antisense strand hybridizes to an mRNA molecule under physiological conditions, wherein the DNA sense strand hybridizes to the RNA molecule under physiological conditions, and wherein a blocking agent is attached to the DNA antisense strand.

37.-38. (canceled)

39. A composition comprising a physiologically acceptable carrier and a component selected from the group consisting of:
(a) the isolated polynucleotides of claim 1; and
(b) the DNA-DNA hybrids of claim 30.

40. A method for reducing the expression of a gene, comprising introducing an isolated polynucleotide of claim 1 or a DNA-DNA hybrid of claim 30 into a cell.

41.-44. (canceled)

45. A method for treating a disease, comprising introducing the isolated polynucleotide of claim 2 into a cell, wherein overexpression of the mRNA is associated with the disease.

46. (canceled)

47. A method of treating an infection in a patient, comprising introducing into the patient the isolated polynucleotide of claim 2, wherein the isolated polynucleotide inhibits entry, replication, integration, transmission, or maintenance of an infective agent.

48. A method for identifying a function of a gene, comprising:
(a) introducing into a cell the isolated polynucleotide of claim 2, wherein the isolated polynucleotide inhibits expression of the gene; and
(b) determining the effect of step (a) on a characteristic of the cell, thereby determining the function of the gene.

49. The method of claim 48, wherein the method is performed using high throughput screening.


SEQUENCE LISTING (2 sequences)

SEQ ID NO: 1 - DNA, 21 bp, Artificial Sequence
  Sense strand from exemplary DNA-DNA hybrid of the invention
tcgaagtattccgcgtacgtt

SEQ ID NO: 2 - DNA, 21 bp, Artificial Sequence
  Template strand from exemplary DNA-DNA hybrid of the invention
tcgaagtattccgcgtacgtt